(19) United States
(12) Patent Application Publication
(10) Pub. No.: US 2005/0070857 A1
(43) Pub. Date: Mar. 31, 2005

(54) Dry & free

(76) Inventor: Kelly Courns, Parker, CO (US)

Correspondence Address: Kelly Courns, 16299 Hawkstone Place, Parker, CO, 80134, US

(21) Appl. No.: 10/674,078

(22) Filed: Sep. 26, 2003

(51) Int. Cl. A61F 7/00; A61F 13/15; A61F 13/20

(52) U.S. Cl. 604/312

(57) ABSTRACT

“Dry&Free” is an underarm deodorant pad for people who suffer from hyperhidrosis. The bottom is sticky so that it can be placed in the underarm area inside a shirt and stay firmly in place. The pad will be placed right underneath the armpit to absorb the excessive sweat. The outside of the pad will contain deodorant/antiperspirant to prevent odor.

### CROSS-REFERENCE TO RELATED APPLICATIONS

Not Applicable

### STATEMENT REGARDING FED SPONSORED R&D

Not Applicable

### REFERENCE TO SEQUENCE LISTING, A TABLE, OR A COMPUTER PROGRAM LISTING APPENDIX

Not Applicable

### BACKGROUND OF THE INVENTION

Not Applicable

### BRIEF SUMMARY OF THE INVENTION

My invention “Dry&Free” is a product for excessive sweating or hyperhidrosis. It is a thin, cotton sheet containing odor-absorbing substances and antiperspirants with an adhesive (sticky) backing that will be placed in the armpit area of shirts to absorb sweat and prevent underarm odor.

People who suffer from hyperhidrosis purchase strong deodorants to prevent odor and excessive sweating, but the problem with existing products is that once a person begins sweating the deodorant comes off. Once the deodorant is gone, there is no longer the odor protection. With “Dry&Free” people can sweat excessively and it won't affect the odor fighting substances and antiperspirants contained in the thin cotton sheet. No matter how much they sweat, they will be protected because the cotton sheet will be stuck to the inside of the shirt of their underarm where it will be in close connect to their armpit. And they won't have to worry about embarrassing wet stains in their armpit area since their sweat is being absorbed into the cotton sheet.

### BRIEF DESCRIPTION OF THE DRAWINGS

None filed

### DETAILED DESCRIPTION

“Dry&Free” is an improvement over existing products for hyperhidrosis. Deodorant and an antiperspirant will be applied to the outside layer of a thin, cotton sheet with an overall thickness of approximately five sheets of paper. The cotton will absorb sweat from the armpits at the same time the top layer containing the deodorant/antiperspirant will provide the needed odor protection. An adhesive will be applied to the bottom of the thin, cotton sheet so that it will stay firmly in place—in the underarm area of the inside of a shirt. When an individual places “Dry&Free” inside his shirt, he will first press his hand firmly underneath his armpit so that the deodorant/antiperspirant rubs off into his armpit.

Essentially “Dry&Free” is an underarm deodorant pad or liner. It will hopefully prevent unwanted stains and provide all day odor protection.

1. What I claim as my invention is an odor-preventing, sweat absorbing thin cotton sheet (deodorant/antiperspirant pad) to be placed in the underarm area inside shirts for people who sweat excessively in their armpits.
